(19) United States
(12) Patent Application Publication
(10) Pub. No.: US 2024/0224910 A9
(43) Pub. Date: Jul. 11, 2024

(54) METHOD, SYSTEM AND USE FOR GERMINATION AND HANDLING OF A SOMATIC PLANT EMBRYO

(71) Applicant: SweTree Technologies AB, Umeå (SE)

(72) Inventors: Malin Abrahamsson, Vattholma (SE); Magnus Hertzberg, Umeå (SE); Ravi Shah, Umeå (SE); Magnus Eriksson, Umeå (SE); Batu Akan, Västerrås (SE); Bengt Peterson, Västerås (SE); Tommy Stenberg, Umeå (SE); Johan Skaborn, Uppsala (SE)

(21) Appl. No.: 18/277,347

(22) PCT Filed: Feb. 15, 2022
(86) PCT No.: PCT/SE2022/050166

§ 371 Date: Aug. 15, 2023

(30) Foreign Application Priority Data
Feb. 15, 2021 (SE) 2150164-8

(51) Int. Cl. A01H 4/00

(52) U.S. Cl. CPC A01H 4/005

(57) ABSTRACT

The present invention relates to methods and systems for obtaining a plantlet from a plurality of somatic plant embryos, and to the use of a compressible and resilient solid substrate comprising a plurality of hydrophilic and biodegradable polymer fibres, as a germination substrate in a method for germination of a somatic plant embryo.

### BACKGROUND

Somatic embryogenesis is a vegetative propagation technology, which makes it possible to mass-produce genetically identical individuals through an asexual reproduction of a source explant. This propagation technology is generally a multi-step process.

Challenge in utilising somatic embryogenesis is the development of cost effective and scalable methods of somatic embryo-based plant production to produce autotrophic and acclimatised plantlets. Several methods and automation processes has been presented for mass production of conifer plants by somatic embryogenesis. There has also been attempts to mass produce other plant species, such as coffee plants and sugar cane, using the somatic embryogenesis process.

Common to many presented automation methods is the handling of mature somatic embryos, where mature somatic embryos must be germinated to produce autotrophic plantlets. This is traditionally done in two steps first with a transfer to a germination media and then with sub-sequent transfer of the individual germinants from a germination media to a media for plantlet growth.

The somatic embryogenesis technology allows for fast and cost-efficient deployment of plants from the breeding front. The somatic embryogenesis technology also allows for clonal or varietal mixture plantation programs. The primary advantages of clonal or varietal mixture forestry and agriculture are the ability to use the best full sibling sib families and additionally the best individuals within such full-sib families for deployment. The somatic embryogenesis technology allows also for predictable production of planting material disregarding bad weather and pest attacks giving variation in seed production and need for pesticide use, as well as reducing land use for planting material production.

In general, in the laboratory, all steps in the process take place in small volumes in petri dishes where the transfer of the SE cultures or mature plant embryos between the petri dishes requires a lot of manual work.

The proliferation step can take place, for most species, in either petri dishes with semi-solid medium or in liquid medium in suspension flasks. Proliferation in liquid medium makes it possible to scale up the production of plant embryogenic tissue in bioreactors. The bioreactors are highly automated and no manual sub-culturing is therefore required.

The maturation step, where the early plant embryos differentiate and develop further into fully mature plant embryos, can for most species take place only in petri dishes with semi-solid medium. Improvements of the culture medium have, however, eliminated the need for sub-culturing which has in turn made it possible to mature plant embryos in larger volumes in boxes.

The mature plant embryos on a petri dish or in a large box are embedded with plant embryogenic tissue. To singulate and prepare the mature plant embryos for the selection step, it is necessary to disperse and separate the mature plant embryos from each other and the non-developed plant embryogenic tissue and callus.

In the laboratory, the separation of the mature plant embryos from the plant embryogenic tissue is done manually with forceps simultaneously with the following steps, selection and deposition. However, separate, select, and deposit large quantities of plant embryos manually is extremely labour intensive and not a possible solution for large scale plant production.

A fluidics system (or disperser) enables dispersion or separation of large quantities of mature plant embryos and plant embryogenic tissue. The mature plant embryos together with the plant embryogenic tissue, from the petri dishes or maturation boxes, are introduced into the disperser. The disperser consists of tubes with varying cross-sectional area and constrictions. When the mature plant embryos together with the plant embryogenic tissue end up by the final cross-sectional area, the plant embryos and plant embryogenic tissue are completely disassociated and dispersed, but still mixed.

In the laboratory, the selection and deposition are done manually, and the selected plant embryos are placed in oriented position on semi-solid germination medium in a petri dish.

The process to produce autotrophic plantlets from mature somatic embryos of conifer woody plants, thus include the following steps, see FIG. 1A, (see e. g. (von Arnold et al., 2008) and (Belmonte, M. F. et al., 2005).

Firstly, high quality mature somatic embryo is placed in a container with controlled air humidity for an optionally desiccation step FIG. 1A [1]. When the selection of high-quality somatic embryos is done manually it needs a well-trained person. The selection can also be done automatically, which can be done by several different quality criteria, as presented in U.S. Pat. Nos. 7,881,502 and 9,053,353.

Partial drying/desiccation of somatic embryos at a slow rate under high relative humidity has been shown to improve the germination and conversion into plants in conifer species. This process mimics the natural process that occurs in seed. It has been also shown that the improvement in germination and conversion rate in somatic embryogenesis in conifers is species/genotype specific. Practically, the desiccation step is carried out by placing somatic embryos in a 6 cm petri dish which in turn is placed in outer bigger 9 cm petri dish with a relative humidity of at least 95%, under sterile condition. The smaller, inner petri dish is left open. The high relative humidity is obtained by having free flowing sterile water in the outer 9 cm petri dish. Care is taken that the water does not come in direct contact with somatic embryos. This petri dish within a petri dish set up is sealed and embryos are stored for up to 3 weeks under dark condition at 20° C., after which they are ready for the germination step.

Secondly, the high quality mature somatic embryo is placed on germination medium FIG. 1A [2] containing an energy source such as sucrose. The germination medium is mostly a sterilised semi-solid media, gel, gelrite and similar. The placement can be done manually or using different robot settings. The mature somatic embryo is then incubated under sterile conditions, in vitro, during a few weeks until root and cotyledons are more developed, optionally the first needles/leaves have developed, FIG. 1A [S3].

Thirdly, the germinant is transferred, FIG. 1A [3], to a pot with a growth substrate, in most cases peat is used. The germinant is left to grow for a few weeks to develop into an established plant that can be further transplanted, FIG. 1A [S4].

Finally, depending on size of the plantlet, it might be transplanted, FIG. 1A [4], to a larger pot before it can be placed in an indoor or outdoor growth location, or stored in a cold room for in-wintering.

Depending on, if a desiccation step [1]-[2], is included or not, in current protocols, all steps require manually handling of individual plant embryos and germinants, which is extremely laboursome and costly for large scale production.

While the above steps are described for conifer species, the steps are essentially the same also for other species.

In the germination step, there must be an energy source to support germination, since the mature somatic embryo lack photosynthesis capacity, i. e. it is not autotrophic. A commonly used energy source supplied in the germination medium is sucrose other energy sources such as maltose have also been tested. A problem with sucrose or any other sugars in the germination medium is the high susceptibility to infection by pathogens, such as fungi or bacteria after transfer to non-sterile conditions, ex vitro, which can result in death or retarded growth of the mature somatic embryos. The germination step is thus preferably done in vitro.

US2005/0124065, Fan et al., suggest a method where the initial steps can be done ex vitro in non-sterile conditions.

In WO2005120212, the susceptibility to infection by pathogens problem is discussed, but not solved. In this document, the germination is done by placing a piece of gel with a plant material in the gel or placing the plant material on top of a gel piece, where the gel medium comprises sugars. The gel medium is placed on top of a sowing substrate, such as peat. The piece of gel with the plant material comprises water, salts (minerals) and sugars. It is recommended that the content of sugars in the substrate is lower than in the carrier preparation, which indicate the problem with sugars.

Only after autotrophy has been reached the somatic seedlings can be removed from the sterile conditions and then transplanted into a non-sterile propagation environment. Even though the art taught by such methods may be practised to produce somatic plantlets, such methods are labour-intensive and bear characteristics of low efficiency, high cost and impracticability for mass production of somatic plantlets in a nursery environment.

Germinants and plantlets are handled in pots of different shapes and made of different materials. These pots can be arranged in trays with hundreds of pots for easily handling, “planting trays”. In nurseries, when seeds are planted, pots are mostly filed with peat as the main rooting substrate, other materials such as stone-wool or foam of different types are also used. Under some circumstances the pots might be covered with some sort of toping such as Styrofoam beads or vermiculite.

Transfer of bare rooted germinants into growth substrate such as peat is normally done ex vitro i.e. not in sterile conditions. The problems with using this procedure are first associated with high costs for multiple handling of the bare rooted germinants, as automation of this step is proved to be very difficult and not applicable on a large-scale operation. Second, the stress endured by the germinants at planting will drastically affect their establishment as plants. This stress is both due to the poor control of water loss from the germinants prior root establishment into the new substrate, and due to the frequent physical damage of the tap root during planting. This damage often leads to the formation of curved taproot, or J-root, a root deformity that has been associated with reduced growth in the field.

Several patent documents have identified the problem with the plant establishment step, which involve germination and root penetrate into the growth substrate to get well anchored plants for the transplanting step and further plant growth, but not solved the problem.

WO99/65291 and WO99/65293, describe rooting substrates such peat, perlite, foam, Oasis®, vermiculite and pumice in different combinations. The covering material tested is coir. Nothing is discussed about how the root is anchored in the substrate.

US 2003/0061639 discusses a process where a somatic embryo is placed onto a substrate and the substrate with the somatic embryo is placed into a growth chamber where water and nutrients are added regularly. The growth substrate can be covered with the same type of rooting substrate or a different material preferred is coir. This patent has limited information about rooting problems.

In WO2006118962 the somatic embryogenesis process for large scale handling of somatic embryos prior to germination and an apparatus to do this is described. Different root shapes are discussed. Curved and coiled roots were classified as non-plantable seedlings. Nothing is taught about how to solve the anchoring of the root in the substrate.

EPA0219133, present a container holding the culturing media, foam, under sterile conditions where plant embryos can germinate. After germination they are covered with soil or potting mix.

None of the above-mentioned documents have solved, or discuss how to solve the problem with rooting, anchoring or the needed or handling the sugar in one approach.

WO2016098083 presents a mechanical device which places germinated somatic embryos in a tube in an oriented direction. In WO2015097603 a device is shown where a tube with a germinated somatic embryo is planted in peat. Together these two patent applications present a mechanical solution to transferring of bare rooted germinants into growth substrate such as peat, the major drawback is a complicated mechanical solution, which must work under sterile conditions. Thus, there is a need for a less complicated solution.

In U.S. Pat. No. 7,207,139 by McKinnis et al. a solution of picking a mature somatic embryo by a vacuum device is presented. It also presents parts for releasing the picked mature somatic embryo. Nothing it taught in what direction the released mature somatic embryo should be placed in a substrate.

US2012/0003074 also present a picking—releasing device for somatic embryos using vacuum and compressed air, which is intended to be used for artificial seeds. The plant somatic embryo is deposited into a cavity oriented so that the cotyledon end is inserted first.

Both US2005/0114918 and WO2018/070866 present a tweezer set-up for picking and releasing devices for somatic embryos. The first one uses one imaging step and the second uses two cameras for image analysis.

In US2005/0114918 the plant somatic embryo is released by a puff of air pressure expelled out of the tip opening to overcome the surface tension and to force the plant embryo out of the vacuum head and deposited into a cavity oriented so that the cotyledon end is inserted first in a substrate.

WO2018/070866 is silent about the orientation of the somatic embryo during placement in a compartment in a tray.

Thus, there is need for a germination substrate where plant embryos are germinated well with well anchored roots; and where the germinant can develop to an established and growing seedling in the same substrate. Furthermore, there is need for a method where a plant embryo is placed in a germination substrate, such that the plant embryos can germinate well with well anchored roots to develop to an established and growing plantlet or plant.

### SUMMARY OF THE INVENTION

The present inventors have identified a need for highly automated methods and corresponding systems to handle the transfer of plant embryos to a suitable germination substrate, and that such automation can be advantageously implemented using growth substrates that are compressible and resilient. The present inventors have also identified that germination substrates comprising a hydrophilic polymer fibre can mitigate or solve problems with insufficient anchoring of roots and mitigate or solve the problem with infections when transferring from sterile to non-sterile conditions. These substrates allow for desiccation, initial germination and further plant growth in the same substrate without handling of individual germinants by transferring them from a germination substrate to plant growth substrate.

Thus, in one aspect, the invention relates to a method for obtaining a plantlet from a plurality of somatic plant embryos by germination of a somatic plant embryo, comprising
Bringing an opening of a nozzle of an embryo transfer device connected to a sub-pressure generator into contact with a plant embryo within the plurality of somatic plant embryos,
Applying a sub-pressure to said opening to thereby attach the plant embryo to the nozzle by way of suction;
Classifying the plant embryo as viable or non-viable;
Providing a germination substrate;
Optionally preparing a well in the germination substrate;
Transferring the plant embryo to the germination substrate if classified as viable, optionally to said well; and
Incubating said germination substrate under conditions suitable for somatic embryo germination, thereby obtaining a plantlet.

In one embodiment, the classification step comprises capturing a digital image of at least one embryo and using a Machine Learning or an image analysis algorithm to classify the embryo as viable for use in subsequent steps of the method.

In one embodiment, the transfer of the plant embryo to the germination substrate comprising bringing the plant embryo into contact with the germination substrate before detaching the plant embryo from the embryo transfer device.

In one embodiment, the method comprises bringing the plant embryo into contact with the germination substrate at an angle of 0°-30°, preferably 0°-10°, to a vertical axis.

In one embodiment, the method further comprises the steps
Adding germination medium to the germination substrate; and
Washing out the germination medium after 1 week or more, such as after 2 weeks or more, such as after 15 weeks or less, such as after 10 weeks or less, such as after 6 weeks or less.

In one embodiment, the method further comprises transferring the germination substrate with the plantlet to a soil.

In one embodiment, the method comprises transferring the plant embryo to a well in the germination substrate with the root forming end down and in contact with substrate, wherein the well optionally has a diameter of 2-6 mm and a depth of 3 mm or more.

In one embodiment, the diameter of the well is reduced after transfer of the plant embryo to the well, such as by allowing the surrounding germination substrate to expand or by applying pressure to at least part of the surrounding germination substrate.

In one embodiment, the method further comprises at least one of the following additional steps
desiccating the embryo on the germination substrate prior to incubations under conditions suitable for somatic embryo germination;
adding a granular top-dressing material on top of the germination substrate to at least partially cover the plant embryo;
Removing excess fluid from the selected plant embryo by suction through the nozzle of the embryo transfer device; and
Growing and acclimating the plantlet ex vitro in the solid germination substrate

In one embodiment, the method comprises the use of a compressible and resilient solid substrate comprising a plurality of hydrophilic and biodegradable polymer fibres as a germination substrate.

In some embodiments, the solid substrate can be compressed at least 3 mm by applying a pressure of less than 0.08 N/mm², such as at least 3 mm by applying a pressure of less than 0.04 N/mm².

In some embodiments, the solid substrate is free of loose adhesive material, and/or wherein the solid substrate is substantially free of particulate matter of less than 0.015 mm width and 2 mm length.

In some embodiments, the solid substrate has a water retention capacity of 0.3-1.2 cm³, such as 0.5-1.2 g/cm³, preferably 0.7-1.0 g/cm³.

In some embodiments, the polymer fibres are non-porous.

In some embodiments, the polymer fibres are constituted of polylactic acid.

In some embodiments, the compressible and resilient solid substrate is used as a desiccation substrate and a germination substrate.

In a further aspect, the invention relates to a system for obtaining a plantlet from a plurality of somatic plant embryos, comprising
An embryo holding container configured to hold a plurality of plant embryos;
An embryo selection unit configured to classify a plant embryo of the plurality of plant embryos as viable or non-viable;
An embryo transfer device connected to sub-pressure generator with a nozzle having a tip surface comprising an opening in fluid contact with the sub-pressure generator; and
At least one germination substrate
   wherein the embryo transfer device is arranged on a robotic arm configured to bring the opening of the nozzle in contact with a plant embryo to facilitate initial attachment of the plant embryo to the nozzle through application of a sub-pressure to the opening, and transfer a plant embryo classified as viable from said embryo holding container to said germination substrate.

In one embodiment, the selection unit comprises a device for capturing a digital image of the plurality of plant embryos and a computer having installed thereon a Machine Learning unit trained to select a viable plant embryo or an image analysis algorithm configured to select a viable plant embryo.

In one embodiment, the embryo transfer device is configured to bring the plant embryo into contact with the germination substrate at an angle of 0°-30°, preferably 0°-10°, to a vertical axis.

In one embodiment, the nozzle tip has an outer diameter of less than 5 mm.

In one embodiment, the nozzle opening is provided within a groove in the nozzle tip surface, wherein said groove is adapted to accommodate a somatic plant embryo of a plant species with which the system is configured to be used.

In one embodiment, the germination substrate is a compressible and resilient solid substrate comprising a hydrophilic and biodegradable polymer fibre as defined in the method aspect of the invention as described above.

In one embodiment, the compressible and resilient solid substrate is used as a desiccation substrate and as a germination substrate.

In a further aspect, the present invention relates to the use of a compressible and resilient solid substrate comprising a plurality of hydrophilic and biodegradable polymer fibres, as a germination substrate in a method for germination of a somatic plant embryo.

In one embodiment, the solid substrate can be compressed at least 3 mm by applying a pressure of less than 0.08 N/mm², such as at least 3 mm by applying a pressure of less than 0.04 N/mm².

In one embodiment, the solid substrate is free of loose adhesive material, and/or wherein the solid substrate is substantially free of particulate matter of less than 0.015 mm width and 2 mm length.

In one embodiment, the solid substrate has a water retention capacity of 0.3-1.2 cm³, such as 0.5-1.2 g/cm³, preferably 0.7-1.0 g/cm³.

In one embodiment, the polymer fibres are non-porous.

In one embodiment, the polymer fibres are constituted of polylactic acid.

In a further aspect, the invention relates to a method for germinating a somatic plant embryo comprising placing a somatic plant embryo in or on a germination substrate comprising or consisting of a compressible and resilient solid substrate comprising a plurality of hydrophilic and biodegradable polymer fibres.

In embodiments, the method comprises the use of a germination substrate as defined in the above described aspect of the invention.

In a further aspect, the invention relates to a method for obtaining a plantlet from a somatic plant embryo, comprising
Placing a mature somatic plant embryo on a compressible and resilient solid substrate comprising a plurality of hydrophilic and biodegradable polymer fibres;
Desiccating said somatic plant embryo;
Adding a germination medium to the solid substrate; and
Incubating the desiccated somatic plant embryo on the solid substrate under conditions suitable for somatic embryo germination, thereby obtaining a plantlet.

In some embodiments, the method according to this aspect utilizes a a compressible and resilient solid substrate comprising a plurality of hydrophilic and biodegradable polymer fibres, as described above.

### BRIEF DESCRIPTION OF THE DRAWINGS

FIG. 1: A. Overview of SE process for woody plants according to prior art

FIGS. 1: B and C show somatic plant embryos and indicate how certain lengths can be measured.

FIG. 2: Illustration of problem with inadequate root anchoring.

FIG. 3: Flow chart of method

FIG. 4: Illustration of the system according to one embodiment of the invention.

FIG. 5: Illustration of a nozzle according to one embodiment of the invention.

FIG. 6: Illustration of a nozzle tip according to one embodiment of the invention.

FIG. 7: Illustration of a nozzle according to one embodiment of the invention.

### DEFINITIONS

Germination Means the development of a shoot and root from a seed embryo or from an artificial plant embryo such as somatic embryos. After germination is completed the plant is autotrophic. The roots of a germinating conifer embryo prefer dark for good development. At the same time, cotyledons need light to start to develop photosynthesis. A somatic plant embryo has started to germinate as soon as the cotyledons has turned into green.

Germination substrate Any substrate that seeds or somatic embryos can germinate in. For somatic embryos it can be agar, gelrite, peat, rock wool, glass wool, coconut fibre, peat, hemp fibres, purane foam, potting compost and/or cellulose wadding or similar substrate. The advantage of these substrates is that they are particularly suitable for cultivating biological material due to their inert properties.

Germination media Germination media comprises, minerals, nutrients, vitamins, and sucrose. Sucrose is needed in the germination media since the somatic embryos lack photosynthesis capacity i. e. is not autotrophic.

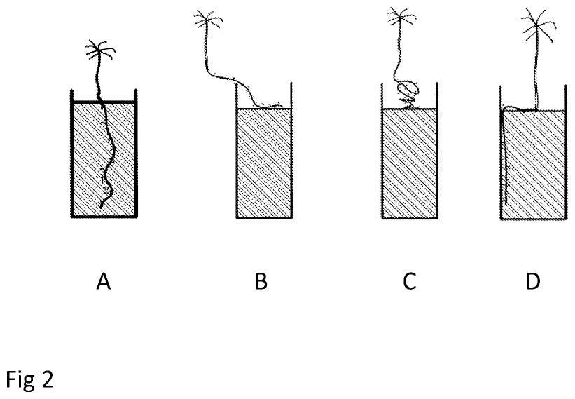

Anchoring of root A root that penetrates the germination substrate during germination and development is said to be anchoring in the germination substrate. This is illustrated in FIG. 2A.

A root that does not penetrate the germination substrate during germination and development is said to be not anchoring in the germination substrate. FIGS. 2B, 2C and 2D illustrate different typical situations where the root is not anchored. The root may grow along the surface of the germination substrate, pushing the plantlet out of the pot (FIG. 2B). The root may get twisted into a bundle on top of the germination substrate, pushing the plantlet up (FIG. 2C). The root may grow downwards outside the germination substrate (FIG. 2D). In the situations in FIGS. 2B, C, and D, the root does not penetrate the substrate and does not get anchored in the germination substrate.

Water Retention Capacity is defined as the capacity of a material to retain water. It is expressed in weight of water per volume of material, e.g. g/cm³. To measure the Water Retention Capacity, a piece of the material of known dry weight (W_{d}) and volume (V) is soaked in a surplus of water and then left in room temperature for one hour on a mesh to allow water to drain and then weighed with retained water to obtain a wet weight (W_{w}). The Water Retention Capacity is calculated as (W_{w}-W_{d})/V.

### DETAILED DESCRIPTION OF THE INVENTION

The present inventors have identified a need for highly automated methods and devices to handle the transfer of plant somatic embryos to a suitable germination substrate, and that such automation can be advantageously implemented using growth substrates that are compressible and resilient.

The use of somatic plant embryogenesis to produce fully developed plants is a highly complex and expensive procedure. The present inventors have developed the present methods and systems in consideration of a high number of conditions that need to be considered and met in order to achieve a high degree of not only successful germination, but also a high relative number of somatic plant embryos that develop into mature and healthy plants that can grow in a natural environment. This is important, particularly when the methods and systems are used to produce large amounts of crop plants or woody plants for commercial use in agriculture, horticulture, and forestry. Such conditions are also partially in conflict with each other, adding to the complexity.

For a plant somatic embryo to germinate and develop into good plantlets the somatic embryo needs:
Light to start the photosynthesis.
The germinating root however prefer darkness.
Nutrients
Sugar needed during initial germination; the plant embryo does not have the capacity to make sugar as it lacks photosynthesis capacity.
A soft substrate, as the growing root is weak.
Well aeriated substrate, as the root needs oxygen,
Water, but the root will not develop well if it is drowned.

It is also preferable that the substrate is inert in the sense that it does not disintegrate when germination medium is added, or release material that negatively affect the germination process.

Furthermore, it is also preferable that the substrate used, is a substrate that both allows initial germination and further plant growth in the same substrate. An advantage with such substrate, is that there is no need of transplanting the germinated plant, which reduces the number of steps in a manual or an automation setup. It is also preferred that the substrate used is a substrate that allows for desiccation, initial germination and further plant growth on and in the same substrate, reducing the steps in a manual or an automation setup even further.

In a method implemented at an industrial scale, it is also preferable that the germination substrate is easy to handle and that the germinated plantlet does not need to be extracted from the substrate at any time. Thus, it is preferable that the substrate works well both under sterile conditions, in vitro, and non-sterile, ex vitro, conditions and that it can be sterilized without negatively impacting the relevant properties. As media added to the germination substrate under sterile conditions may be a nutrient source for pathogenic microorganisms under non-sterile conditions, it is also preferable that such germination media can be washed out of the germination substrate on transfer from sterile to non-sterile conditions. The germination substrate should preferably also have a mechanical strength that makes it easy to transfer the germination substrate from one location to another with a plantlet rooted in the germination substrate.

It has unexpectedly been found that when transferring plant embryos to a germination substrate, significantly improved results in terms of successful germination can be achieved if the plant embryo is pushed against the germination substrate in a substantially vertical alignment to provide close physical contact between the plant embryo and the germination substrate, with the root forming tip pointing down, and embedded within the germination substrate. However, the limits of precision of both human and robotic arms in combination with the small dimensions of plant embryos entails a high likelihood that a plant embryo transfer device mounted on a robotic arm pushes the plant embryo against the germination substrate with some force. The use of conventional germination substrates therefore risks crushing the plant embryo against the germination substrate as it is placed in or on the germination substrate, as conventional germination substrates are usually quite hard, i.e. non-compressible at the forces at hand in an automated system. Simply put, the plant embryo is at a significant risk of getting crushed between many conventional germination substrates and a nozzle of an embryo transfer device.

Furthermore, the use of a resilient germination substrate, i.e. a germination substrate that returns to its previous shape, facilitates convenient embedding of a plant embryo in an automated system. With a resilient germination substrate, a hole or rift can be made in the germination substrate in which the plant embryo is placed, and which hole or rift then closes itself at least in part due to the resilience of the germination substrate or is mechanically closed.

It has been found that germination substrates made from hydrophilic polymer fibres have suitable properties regarding compressibility and resilience for use in an automated system according to the present invention. The present inventors have also found that germination substrates comprising a hydrophilic polymer fibre can mitigate or solve problems with insufficient anchoring of roots in somatic embryo genesis production of plants. Such germination substrates are also sufficiently rigid for transfer of the germination substrate with a developed plantlet to a different location, such as transfer to a tray or planting the germination substrate with the plantlet in a soil for further growth into a fully developed plant. It is a further advantage of such germination substrates that they are biodegradable. This obviates the need to extract the plantlet from the germination substrate before replanting, which almost inevitably risks damaging the newly developed roots of the plantlet. As the germination substrate is biodegradable, it is possible to keep the plantlet in the germination substrate when replanting, and letting the plantlet develop to a plant while the germination substrate gradually degrades in the soil or other growth environment.

An unexpected advantage with this method is that the plant embryos can be germinated and grown in the same substrate, which significantly improved results in terms of successful germination.

The use of a germination substrate comprising hydrophilic polymer fibres also solves the known problem of insufficient anchoring as described above. It has been found that when a plant embryo is placed in a substantially vertical position in a germination substrate comprising hydrophilic polymer fibres, it develops roots that protrudes into the fibrous germination substrate and anchors the developing plantlet to the germination substrate. This is a huge advantage over other combined germination substrates where a substantial number of plantlets do not anchor in the germination substrate but rather develop roots on, or very shallow in, the top surface of the germination substrate, or is simply pushed up from the germination substrate out of the planting tray or other container holding the germination substrate.

The use of a compressible and resilient solid germination substrate comprising a plurality of hydrophilic and biodegradable polymer fibres as a germination substrate thus facilitates both manual and automated methods for germination and continued plant growth of a somatic plant embryo.

The present invention thus in one aspect relates to the use of a compressible and resilient solid germination substrate comprising a hydrophilic and biodegradable polymer fibre, as a germination substrate in a method for germination and continued plant growth of a somatic plant embryo.

In one embodiment, the compressibility of the solid germination substrate is such that it can be compressed at least 3 mm by applying a pressure of less than 0.08 N/mm², such as at least 3 mm by applying a pressure of less than 0.04 N/mm². As the solid germination substrate should also be resilient, it will return to near its original shape after being subjected to such pressure. It may return to its original shape within 10 minutes, within 5 to 10 minutes, or within less than 60 seconds, such as within less than 30 seconds or less, with no permanent depression.

In one embodiment, the compressed germination substrate might be returned to its original shape mechanically, which might be done with a tool such as a forceps.

In one embodiment, the solid germination substrate is free of loose adhesive material, before addition of germination medium. Such material may otherwise adhere to parts of a plant embryo transfer device that are intermittently in contact with the germination substrate. Such contaminations might originate from semi-solid gel, peat or the similar. Such contamination of parts of the plant embryo transfer device would necessitate cleaning of those parts between plant embryo transfers which would complicate both methods and systems used for performing methods. Preferably, at least the parts of the germination substrate that in use may be in contact with the plant embryo transfer device are free of such loose adhesive material. Adhesive properties of any loose material present in the germination substrate are preferably assessed vis-à-vis the material in the parts of the plant embryo transfer device that in use may be in contact with the germination substrate.

In one embodiment, the solid germination substrate is substantially free of particulate matter of less than 0.015 mm width and 2 mm length. Such material may arise as dust particles in a fibrous germination substrate when it is cut, e.g. when it is cut into pieces of suitable size for use as germination substrates. Such fine particulate matter is advantageously removed before the substrate is used as a germination substrate as it may also adhere to parts of a plant embryo transfer device that are intermittently in contact with the germination substrate.

In one embodiment, the solid substrate has a water retention capacity of 0.3-1.2 cm³, such as 0.5-1.2 g/cm³, preferably 0.7-1.0 g/cm³. This is advantageous when an aqueous germination medium is to be added to the substrate to facilitate germination.

In one embodiment, the polymer fibres of the solid substrate are non-porous. This is advantageous when an aqueous germination medium that has been added to the germination substrate is to be washed out of the germination substrate. When the polymer fibres are non-porous, the wash solution can readily access the entire internal surface area of the germination substrate to facilitate washing in a relatively short time, and aqueous germination medium does not penetrate into the polymer fibres.

It has been found that some substrates used for germination, such as peat, releases substances toxic to plant embryos when subjected to temperatures necessary for sterilisation. As plant embryos are susceptible to infections by plant pathogens, it is highly desirable to be able to sterilise the germination substrate prior to use. Thus, it is preferable that the solid substrate can be subjected to at least one sterilisation method, such as autoclaving and/or irradiation (such as gamma radiation or X-ray radiation), without decreasing its ability to function as a germination substrate.

In one embodiment, the polymer fibres are constituted of polylactic acid. Polylactic acid is an environmentally friendly thermoplastic. The fibre forming substance is a lactic acid polymer in which at least 85% by weight are lactic acid ester units derived from naturally occurring sugars, that may be extracted from e.g. sugar beets and corn.

Although biodegradable, polylactic acid is quite durable in most applications. In fact, PLA does not readily degrade unless it is exposed to prolonged times of high humidity and elevated temperatures 60° C.) which results in rapid decomposition of the fibre.

Polylactic acid fibres are commercially available from Holland BioProducts (Nijmegen, the Netherlands) with the brand name, “Whiteplug Sow”. These plugs consist of a polylactic (PLA) stable fibre with flakes of a super absorbent polymer of poly-potassium acrylic co-polymer applied thereon. Such PLA substrates may be used in the present invention with or without, preferably without, the acrylic co-polymer flakes.

The thickness of the polylactic acid fibres can be about 30 to 70 μm, 30 to 60 μm, 35 to 55 μm or preferably about 40-50 μm (micrometre) in diameter. The fibres might be non-hollow.

Other useful polymers are other biodegradable plastics, such as polyhydroxyalkanoates, naturally produced by various micro-organisms, modified cellulose, such as cellulose esters and cellulose acetate and nitrocellulose, polyglycolic acid (PGA), polybutylene succinate (PBS), polycaprolactone (PCL), poly(vinyl alcohol) (PVA, PVOH). Optionally, plant polymers such as lignocellulosic fibers or lignin-based polymer composites may well work as a germination substrate. This includes substrates comprising, or composed of, coconut fibres and peat, such as 30-50% coconut fibres and 50-70% peat. Such substrates are commercially available from ViViPak B. V., 's-Gravendeel, the Netherlands, under the trade name Obturo®. Substrates comprising peat are preferably sterilised by irradiation, as high temperature treatment of peat may release substances toxic to plant embryos.

In some embodiments, the above described compressible and resilient solid substrate is used as both a desiccation substrate and a germination substrate.

In some embodiments, the above described compressible and resilient solid substrate comprising a hydrophilic and biodegradable polymer fibre, is used as a germination substrate in the method and system according to the invention, as described below.

In one aspect, analogous to the use of a compressible and resilient solid substrate described above, the present invention also relates to a method for obtaining a plantlet from a somatic plant embryo, comprising
Placing a mature somatic plant embryo on a compressible and resilient solid substrate comprising a plurality of hydrophilic and biodegradable polymer fibres;
Desiccating said somatic plant embryo;
Adding a germination medium to the solid substrate; and
Incubating the desiccated somatic plant embryo on the solid substrate under conditions suitable for somatic embryo germination, thereby obtaining a plantlet.

In embodiments, the solid substrate used in the method according to this aspect is as described above.

A desiccation step performed on a compressible and resilient solid substrate as described herein can be performed analogously to desiccation steps as known in the art, such as described in the background section.

In a further aspect, the present invention relates to a method for obtaining a plantlet from a plurality of somatic plant embryos by germination of a somatic plant embryo, comprising:
Bringing an opening of a nozzle of a plant embryo transfer device connected to a sub-pressure generator into contact with a plant embryo within the plurality of somatic plant embryos;
Applying a sub-pressure to said opening to thereby attach the plant embryo to the nozzle by way of suction;
Classifying the plant embryo as viable or non-viable;
Providing a germination substrate;
Optionally preparing a well in the germination substrate, such as by means of drilling;
Transferring the plant embryo to the germination substrate if classified as viable, optionally to said well; and
Incubating said germination substrate under conditions suitable for somatic embryo germination, thereby obtaining a plantlet.

It is noted that the steps are not necessarily performed in the exact order that they are listed above. For instance, the classification of a plant embryo as viable or non-viable may be performed before the plant embryo is attached to the nozzle of the plant embryo transfer device.

In some embodiments, the method according to the invention is performed by the system according to invention as described below. Embodiments of the method described below are applicable mutatis mutandis to the system, and vice versa.

Methods for preparing somatic plant embryos for further development by the present invention are known as such and not part of the present invention. Such methods are described in detail in the references cited in the background part of the present disclosure.

An automated robotic system enables a fast and efficient selection and deposition of the plant embryos. The robot may have the ability to image, tag and select plant embryos of good quality, and deposit the plant embryo into a germination substrate, preferably located in a planting tray that is in turn placed in a larger box.

An advantage with this setting is that a selected and picked plant embryo is picked up and deposited using the same device, a nozzle of the plant embryo transfer device, and there is no need for transfer of the plant embryo between devices.

The germination i.e., when the plant embryo has developed a root and a shoot, takes approximately 6 weeks. The time may vary between plant species.

The germinated plant embryos in the germination substrate in the planting trays, do not need transplanting and can directly be gradually acclimatized to ex vitro conditions. The elimination of the transplanting step reduces the stress on the young plants and also reduces costs.

The classification of a viable plant embryo can be done manually by a laboratory worker experienced in development of plantlets from plant embryos by manual inspection of plant embryos and classifying one or more plant embryos as viable or non-viable based on experience. The classification of a plant embryo as viable or non-viable can also be done by an automated system such a Machine Learning algorithm or image analysis system.

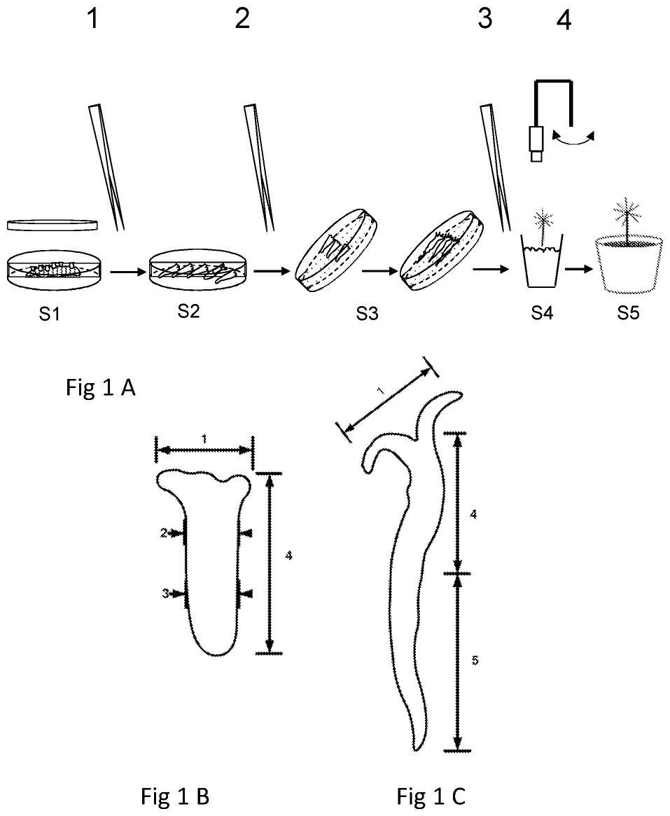

Some quality criteria of somatic embryos are set out in WO2011123038 (incorporated herein by reference). The sides of the hypocotyls of a viable plant embryo should be smooth, and no swelling should be seen in the lower part of the hypocotyl, while a poor-quality somatic plant embryo has a rough surface and is often swollen at the lower end. A high-quality somatic embryo has moderately developed cotyledons with little greenish tone, and no root should be seen. In order, to measure the quality of the somatic embryos, different measurements can be taken, as illustrated in FIGS. 1B and 1C, wherein (1) is the width across the cotyledons; (2) is the width one third of the way down the hypocotyl; (3) is width two thirds of the way down the hypocotyl; (4) is the length of hypocotyl; and (5) is the length of root. These criteria can and will vary between somatic embryos from different plant species. The person skilled in somatic embryogenesis for a specific plant species can point out which plant embryos are likely to produce a plant, and this can be used to train a Machine Learning algorithm to select viable plant embryos from any species.

In poorly developed plant embryos, there is a tendency to constriction in the hypocotyl near the cotyledons and/or a swelling at the base of the hypocotyl, so that the ratio of widths one third down of the hypocotyl (2) to the width across the hypocotyl two thirds down the hypocotyl (3) is below unity, i.e. (2)/(3)<1. In well-developed plant embryos the ratio is just above unity.

In one embodiment, the classification step comprises capturing a digital image of the plurality of plant embryos and using a Machine Learning or an image analysis algorithm to classify plant embryo as viable or non-viable for purposes of subsequent steps of the method.

In one embodiment, the classification is performed by Machine Learning using a neural network. The neural network used is preferably initially trained by the following manual process. Images of plant embryos are taken, and experts manually classify these images according to the appropriate classes, viable and non-viable. The classified images are sorted into two distinct training and validation datasets. The neural networks take the training dataset images as input data and teaches itself the required visual features for classification. The performance of the neural network is then finally tested using the validation dataset before deployment.

When the method is performed repeatedly, the neural networks used in the method could be continuously improved by the means of automatic or reinforced classification. This means that the neural network input data can be extended by using the output results of the method.

In the case of planting suitability, plant embryos that after planting have germinated and developed into a plantlet can automatically be classified as “viable”, and in the same way, plant embryos that have not developed to an appropriate degree can automatically be classified as “non-viable”. The neural network could then continuously be retrained and re-evaluated based on the real-world data.

The neural network may be of any type suitable in a classification task as detailed herein. A currently available machine learning platform suitable for use in the present invention is TensorFlow (www.tensorflow.org).

The method also requires the use of a plant embryo transfer device having a nozzle with an opening connected to a sub-pressure generator. The plant embryo transfer device is preferably mounted on a robotic arm that can be programmed to move the plant embryo transfer device as required to perform the method. Such robotic arms are known in the art and commercially available, e.g. ABB IRB1200 from Asea Brown Boveri, Ltd. Robotic arms, also termed industrial robots, are commercially available from a number of manufacturers. In general, they comprise a number of connected segments that are moveable and rotatable relative each other so as to move a tool mounted on the robotic arm between various positions in space and effect actions with the tool. The movements and rotations of the segments are effected by motors and controlled by a computer (herein and below termed a central processing unit) with a suitable user interface for a user to achieve the desired movements.

Applying a sub-pressure to the nozzle opening facilitates attachment of a selected plant embryo to the nozzle by way of suction. The plant embryos are usually kept in an aqueous storage medium prior to performing the present method, to keep them from drying. Sterile clean water might also be used. Attaching the plant embryo using a sub-pressure might also facilitates draining of surplus medium from the plant embryo. The plant embryo can then, if desired, be contacted with fresh germination medium that may have different composition as compared to the storage medium.

The plant embryo is to be transferred to a germination substrate, which is provided according to the method. In some embodiments, the germination substrate is provided with a well in which the plant embryo is placed. Such a well can be pre-formed in the substrate, or it may be formed just before the plant embryo is to be placed therein. Exemplary ways of providing a well is by drilling, or by inserting two elongate members in contact with each other into the substrate and separating them, thereby creating a rift or crevice in the germination substrate. The well may have a width, breadth, and/or diameter of about 2-6 mm, and a depth of about 1-10 mm, 1-5 mm or 1-3 mm, preferably about 3 mm. In some embodiments, the well is partially or wholly closed after placing the plant embryo in the well. If the germination substrate is made of a resilient material, closure of the well may be achieved by allowing the germination substrate to relax or otherwise expand to close the well partially or wholly. It is also possible to apply pressure to material in the walls of the well to close the well partially or wholly.

The plant embryo is transferred to the germination substrate. In some embodiments, the transfer of the plant embryo to the germination substrate comprises bringing the plant embryo into contact with the germination substrate before detaching the plant embryo from the plant embryo transfer device. That is, the plant embryo is lightly pushed against the germination substrate. Without being bound by theory, it is believed that such forced physical contact connects the plant embryo to the germination substrate in a beneficial way and helps in development of roots that anchor in the germination substrate. In practising the method, a plurality of germination substrates, each intended to receive a single plant embryo, is provided in a planting tray with a number of recesses adapted to receive a single germination substrate. When plant embryos have been placed in or on the germination substrates, the entire planting tray is placed under conditions suitable for somatic embryo germination as further described below.

In one embodiment, the plant embryo is brought into contact with the germination substrate at an angle of 0°-30°, preferably 0°-10°, to a vertical axis. That is, the plant embryo is placed in a substantially standing fashion, with the root-forming end being placed downwards.

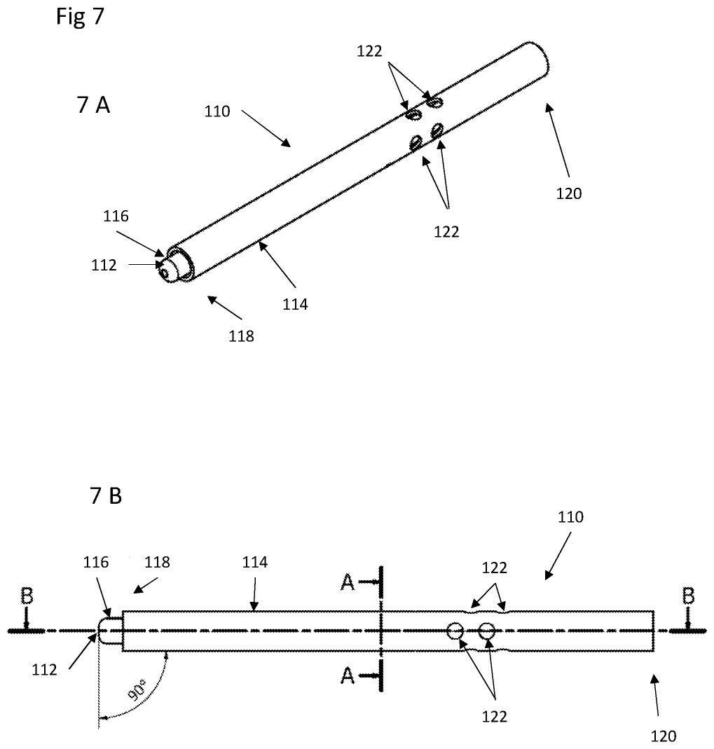

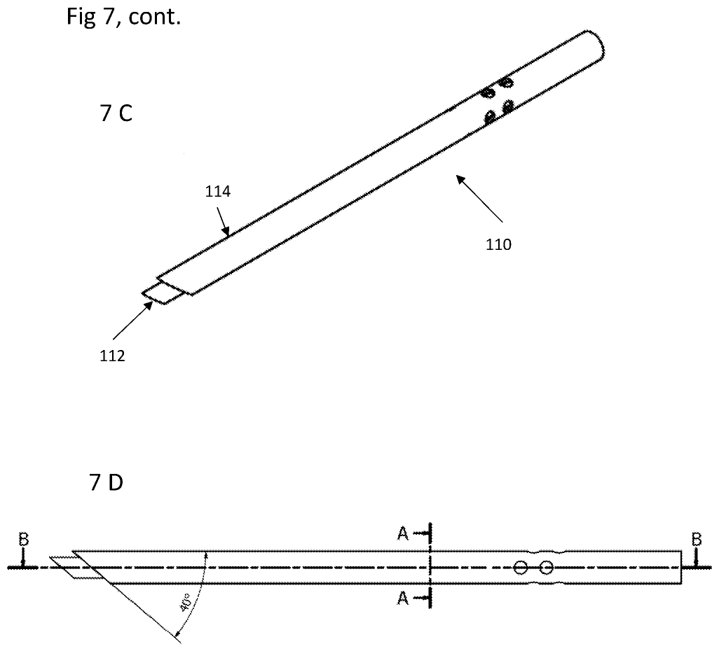

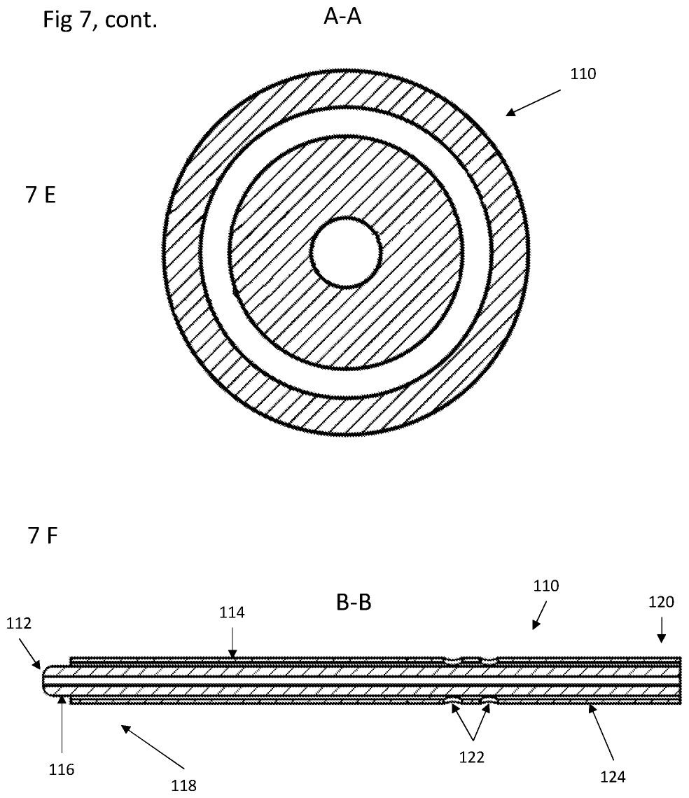

In some embodiments, when the plant embryo is lightly pushed against the germination substrate, it might need a puff of compressed air to be released from the nozzle of the plant embryo picking device. In some embodiments, as further described below in relation to FIG. 7, the nozzle comprises an inner and an outer tube and the puff of compressed air can be provided through the void space between the tubes to push the embryo away from the nozzle tip surface. In other embodiments, the negative pressure used to attach the embryo to the nozzle tip surface is reversed to a positive pressure to release the embryo.

The method further comprises incubating the germination substrate under conditions suitable for somatic embryo germination, thereby obtaining a plantlet. Conditions suitable for somatic embryo germination are known in the art and described i.a. in WO2011123038.

In some embodiments when germination medium is added to germination substrate it is subsequently washed out after 1-15 weeks, 2-10 weeks, or preferably 2-6 weeks. This is done to remove all sugars contained in the germination medium before transferring the plantlet to non-sterile conditions. Residual sugars may promote growth of potentially pathogenic microorganisms and is thus advantageously removed prior to exposing the germination substrate to non-sterile conditions.

In some embodiments, the method comprises transferring the germination substrate with the plantlet to a soil.

In some embodiments, the method comprises desiccating the embryo on the germination substrate prior to incubations under conditions suitable for somatic embryo germination, e.g. substantially as described in Example 5.

In some embodiments, the method comprises adding a granular top-dressing material on top of the germination substrate to cover the plant embryo at least partially.

In some embodiments, the method comprises removing excess fluid from the selected plant embryo by suction through the nozzle of the plant embryo transfer device.

In some embodiments, the method comprises growing and acclimating the plantlet ex vitro in the solid germination substrate.

In some embodiments, the method comprises the use of a compressible and resilient solid substrate comprising a hydrophilic and biodegradable polymer fibre as described in detail above.

In a further aspect, the present invention relates to a system for obtaining a plantlet from a plurality of somatic plant embryos, comprising
A plant embryo holding container configured to hold a plurality of plant embryos;
A plant embryo selection unit configured to classify a plant embryo as viable or non-viable;
A plant embryo transfer device connected to sub-pressure generator with a nozzle having a tip surface comprising an opening in fluid contact with the sub-pressure generator; and
At least one germination substrate
   wherein the plant embryo transfer device is arranged on a robotic arm configured to bring the opening of the nozzle in contact with a plant embryo to facilitate initial attachment of the plant embryo to the nozzle through application of a sub-pressure to the opening and transfer a plant embryo classified as viable from said plant embryo holding container to said germination substrate.

The system, according to the invention is generally adapted to perform the method according to invention as described above. Embodiments of the system described below are applicable mutatis mutandis to the method, and vice versa.

The plant embryo holding container may be any standard or custom-made container made of a suitable material that is compatible with a medium for storage of the plant embryos and provides access to the plant embryos by the plant embryo transfer device.

The plant embryo selection unit may be a dedicated or a general-purpose computer having input means for input of data relating to individual plant embryos indicating position in the plant embryo holding container and data related to expected plant embryo viability, and output means for sending plant embryo position data for an individual plant embryo to a robot including the robotic arm on which the plant embryo transfer device is arranged.

In its simplest form, the plant embryo selection unit obtains through its input means a viability score set assigned to an individual plant embryo by an experienced laboratory worker and positional data for that individual plant embryo. Positional data can in such an embodiment be obtained by comparing the plant embryo's position to a grid, coordinate system, or other positional indicator displayed on the bottom of the plant embryo storage container.

In other embodiments, the plant embryo selection unit comprises a device for capturing one or more digital images of the plurality of plant embryos in the plant embryo storage container and a computer having installed thereon a Machine Learning unit trained to classify a plant embryo as viable or non-viable, or an image analysis algorithm configured to classify a plant embryo as viable or non-viable. The machine learning unit or image analysis algorithm are typically installed as computer executable code on a central processing unit within the plant embryo selection unit. The machine learning unit may be trained by a laboratory worker experienced in selection of viable plant embryos, or by providing the unit with feedback on actual viability of selected plant embryos, or a combination thereof.

In one embodiment, plant embryo selection unit comprises three machine vision camera systems, a primary camera system, a macro camera system and a third camera system.

The primary camera system is configured to capture images of the plant embryo holding container and selecting plant embryos that are suitable for picking and transfer by the plant embryo transfer device. The selection criteria may i.a. include that a plant embryo should not be too close to another plant embryo or to a wall of the container, and that it is possible to determine the orientation of the plant embryo. The primary camera system may perform the following tasks: Detect and localize plant embryo branch sides by the means of a neural network; Find the centre point or a point just below the cotyledons of the detected plant embryos by the means of a machine learning algorithm and/or a classical vision algorithm; Find the orientation of plant embryos based on centre point and branch side positions; Classify the detected plant embryos as “isolated” or “not isolated” by the means of a neural network; Classify the detected plant embryos as “viable” or “non-viable” by the means of a neural network

The macro camera system is configured to capture close-up images of picked plant embryos to verify planting suitability (i.e. images used for classification of plant embryos as viable or non-viable) and to confirm that a plant embryo is indeed picked up by and attached to the plant embryo transfer device. The macro camera system may classify a picked plant embryo as “viable”, or “non-viable”, or “no plant embryo” if there is no plant embryo picked and attached to the nozzle, by the means of a neural network.

The third camera system is configured to capture images of the germination substrate to determine its position and facilitate transfer of the plant embryo to a correct position. The third camera system may perform the tasks: Find the corners of germination substrates by the means of a neural network; Calculate the position of the germinations substrates based on the detected corners.

The plant embryo transfer device is provided with a nozzle having a tip surface with an opening connected to a sub-pressure generator. When sub-pressure is applied and the nozzle is brought into contact with a plant embryo, the plant embryo is attached to the tip surface of the nozzle and can be transferred to the germination substrate. When in position at the germination substrate, the plant embryo can be detached from the nozzle by releasing the sub-pressure or applying a positive pressure. The opening of the nozzle may also be connected to a fluid trap to collect any fluid that is sucked through the opening, such as storage medium on the plant embryo.

The robotic arm on which the plant embryo transfer device is arranged, and the operation of the sub-pressure generator are preferably under the control of a central processing unit within the system. The central processing unit can thus control the robotic arm to position the nozzle of the plant embryo transfer device in close proximity to a selected plant embryo, actuating a sub-pressure resulting in attachment of the plant embryo to the nozzle, control the robotic arm to position the nozzle of the plant embryo transfer device at a germination substrate, and finally release the sub-pressure to detach the plant embryo and place it in or on the germination substrate.

The plant embryo transfer device is preferably configured to be able to bring the plant embryo into contact with the germination substrate at an angle of 0°-30°, preferably 0°-10°, to a vertical axis.

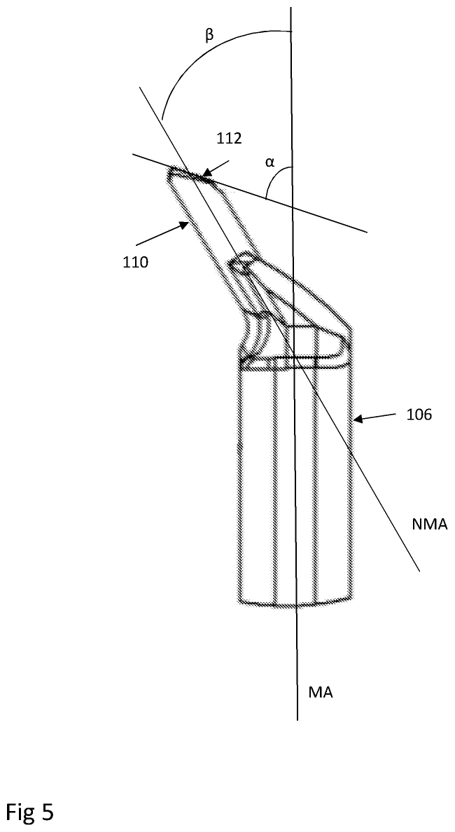

In some embodiments, the plant embryo transfer device extends along a device main axis, and the nozzle extends along a nozzle main axis [MNA] in FIG. 5. at an angle of 20° to 40° to said device main axis, and wherein the nozzle has a tip surface arranged at an angle of 65°-75° to said device main axis [MA] in FIG. 5. This configuration has been found to facilitate an efficient protocol for picking plant embryos in the plant embryo storage container and positioning them on the germination substrate in a substantially standing fashion, with the root-forming end being placed downwards, as described above.

In some embodiments, the nozzle tip has a diameter of less than 5 mm, in order, to facilitate precise operation in attaching plant embryos to the nozzle.

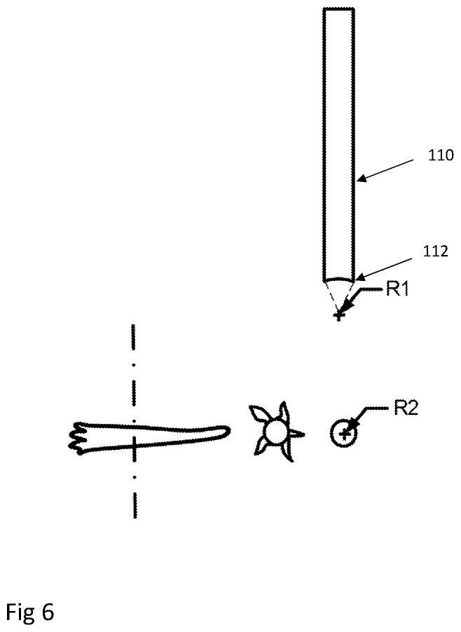

In some embodiments, the nozzle opening is provided with a groove of a depth of 0.5-1.5 mm in the nozzle tip surface [112]. This provides a guiding structure for aligning the plant embryo on the nozzle tip surface, in order, to facilitate precise operation in placing plant embryos in or on the germination substrate, see FIG. 6.

In some embodiments, the nozzle comprises an outer tube and an inner tube protruding from the outer tube and ending with the nozzle tip surface at a distal end of the nozzle and the proximal end of the inner tube being connected to the sub-pressure generator, and the nozzle being configured so that positive pressure can be applied to the space between the outer tube and the inner tube to provide an air stream towards the distal end of the nozzle.

The wall of the outer tube may further be provided with one or more through-holes, as shown in FIG. 7, for application of positive pressure for delivering a puff of compressed air to release an embryo attached to the tip surface.

In some embodiments, the system uses the compressible and resilient solid substrate comprising a hydrophilic and biodegradable polymer fibre described above as the germination substrate.

It has also surprisingly been found that a compressible and resilient solid substrate comprising a hydrophilic and biodegradable polymer fibre can be used as both a substrate in a desiccation step and as a germination substrate. Performing the desiccation step and the germination step reduces the need for transfer of embryos between substrates, thus simplifying the overall process which facilitates automation, and also reduces the risk of damage to the embryos during handling.

In one aspect, the present invention thus relates to a method for obtaining a plantlet from a somatic plant embryo, comprising
Placing a mature somatic plant embryo on a compressible and resilient solid substrate comprising a plurality of hydrophilic and biodegradable polymer fibres;
Desiccating said somatic plant embryo;
Adding a germination medium to the solid substrate; and
Incubating the desiccated somatic plant embryo on the solid substrate under conditions suitable for somatic embryo germination, thereby obtaining a plantlet.

The method according to this aspect may utilize a compressible and resilient solid substrate as described herein. The method may also be performed in a system according to the present disclosure, and the system according to the invention can be configured to implement the method according to this aspect.

The invention will now be further described in relation to the appended drawings.

FIG. 1A is an overview of the general process for obtaining woody plantlets by somatic embryogenesis. FIG. 1A. describes the manual somatic embryo method for creating plantlets from woody plants. The first manual interaction is when [1] the mature plant embryo is isolated from immature plant embryos (S1) and placed horizontally in a plastic container under sterile conditions; the second [2] occur after 3-21 days of desiccation (S2). The desiccation step is optional. Then mature somatic plant embryo is transferred to a gelled culture medium for initiation of germination processes. The germinated somatic embryo will under appropriate culture medium composition and light conditions initiate roots (S3). The third manual transfer [3] is when the germinant having a small or a large root formed is transferred to an upright position with the root in a growth substrate for further plantlet development (S4). The fourth [4] and final transfer is mainly done by a robotic setting is when small plantlet with the growth substrate is transferred to a larger container of growth substrate for further growth to larger plant (S5).

FIGS. 1 B and C show somatic plant embryos and indicate how certain lengths can be measured.

FIG. 2 describes problems with root anchoring, as discussed above in the background section. In the case the germination substrate is too hard or other unfavourable properties of the substrate the growing root cannot penetrate the substrate, which leads to a root, which is not anchored. FIG. 2A shows a well anchored root, which have penetrated the substrate well. FIGS. 2B, 2C and 2D represent different types of root problems. In 2B, the root does not penetrate the substrate and push the shoot out of the pot. In 2C the root forms a knot and will die. In 2D, the root grows along one of the sides in the pot, in this case the plant may grow well, but it is not suitable for automated transplanting. A plantlet with a root according to 2A is well suited for automated transplanting.

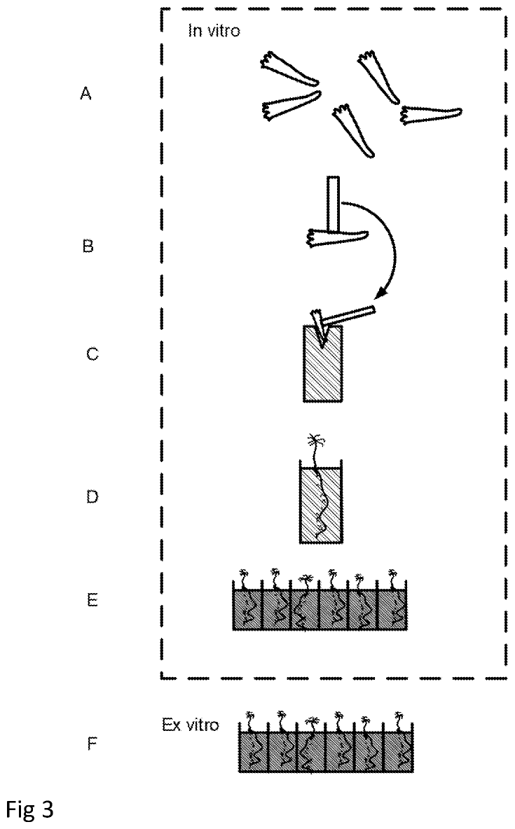

FIG. 3 is a flow chart illustrating the method according to the invention. Step A illustrate selecting of a plant embryo; step B, picking of a plant embryo; step C, deposition of a plant embryo; D, germination of a plant embryo; E, germination of plant embryos in tray and; F, further growth and acclimatisation of the plant. Washing and changing of media can optionally be done one, two or several times, for example between step E to F.

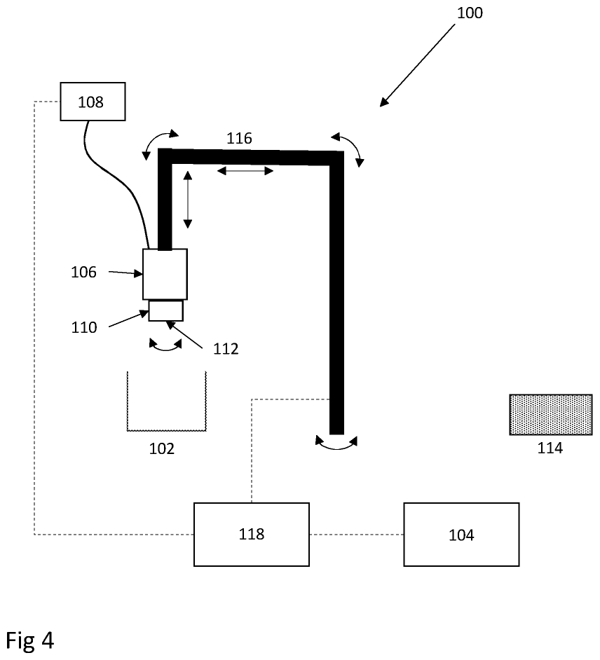

FIG. 4 is a schematic illustration of the system (100) according to the invention. The system (100) comprises a plant embryo holding container (102) configured to hold a plurality of plant embryos; a plant embryo selection unit (104) configured to classify a plant embryo of the plurality of plant embryos as viable or non-viable; a plant embryo transfer device (106) connected to a sub-pressure generator (108) with a nozzle (110) having a tip surface (112) comprising an opening in fluid contact with the sub-pressure generator (108); and at least one germination substrate (114) wherein the plant embryo transfer device (106) is arranged on a robotic arm (116) configured to bring the opening of the nozzle (110) in contact with a plant embryo to facilitate initial attachment of the plant embryo to the nozzle (110) through application of a sub-pressure to the opening and transfer the plant embryo from said plant embryo holding container (102) to said germination substrate (114).

The robotic arm is movable, e.g. as indicated by arrows in the figure, in order to position the nozzle (110) at any position in the plant embryo holding container (102) and at any position on the germination substrate (114) and at any angle to the plant embryo holding container (102) and the germination substrate (114). The movements of the robotic arm is controlled by a central processing unit (118), that preferably also controls operation of the sub-pressure generator (108). The central processing unit may thus transmit and receive data to and from the robotic arm (116), the sub-pressure generator (108), and the plant embryo selection unit (104) through wired or wireless connections (shown in dashed lines).

FIG. 5 is a schematic illustration of one embodiment of the nozzle in an embodiment where the plant embryo transfer device (106) extends along a device main axis (MA), and the nozzle (110) extends along a nozzle main axis (NMA) at an angle (β) of 20° to 40° to said device main axis (MA), and wherein the nozzle (110) has a tip surface arranged at an angle (a) of 65°-75° to said device main axis.

FIG. 6 is a schematic illustration of the outermost tip of one embodiment of the nozzle (110), wherein the tip surface (112) is curved to provide a groove adapted to accommodate a somatic plant embryo. The groove has a curvature of 1/R1, wherein R1 is equal to or larger than the radius (R2) of a cross-section of a somatic embryo of a plant species for which the nozzle (110) is intended to be used. R2 is the radius at the cross-section of a desired plant embryo at the indicated line. This corresponds to the half the width [2] in FIG. 2A.

FIG. 7 is a schematic illustration of a nozzle (110) according to one embodiment of the invention. The nozzle (110) can be detachably mounted into the embryo transfer device (106) (cf. FIG. 5). FIGS. 7A and 7B show an overview of one embodiment of the nozzle (110). In this embodiment, the nozzle (110) comprises an outer tube (114) and an inner tube (116) protruding from the outer tube (114) and ending with the nozzle tip surface (112) at a distal end (118) of the nozzle (110). The wall of the outer tube (114) is provided with one or more through-holes (122). The proximal end (120) of the inner tube (116) is, in use, connected to the sub-pressure generator (108) (cf. FIG. 5). The nozzle tip surface (112) and the tip of the distal end of the outer tube (114) are substantially orthogonal to the nozzle main axis (B-B in FIG. 7B).

FIGS. 7C and 7D show an overview of a further embodiment of the nozzle (110). This embodiment differs from the embodiment shown in FIGS. 7A and 7B in that the nozzle tip surface (112) and the tip of the distal end of the outer tube (114) are at an angle of 40° to the nozzle main axis (B-B in FIG. 7D).

FIG. 7E shows a cross-section of nozzle (110) along the line A-A in FIGS. 7B and 7D.

FIG. 7F shows a cross-section of nozzle (110) along the line B-B in FIGS. 7B and 7D.

As shown in FIG. 7F, a bushing (124) sealing the inner tube (116) to the outer tube (114) is placed between the through-hole(s) (122) and the proximal end (120) of the nozzle. The through-holes (122) may in use be connected to a positive-pressure generator. A positive pressure can be applied to the space between the outer tube (114) and the inner tube (116) via the through holes (122) to provide an air stream towards the distal end (118) of the nozzle (110) to aid in detachment of embryos attached to the nozzle tip surface (112).

### Experiments

The following examples are illustrative of the invention and provided for the skilled reader to fully understand the invention. They are not to be construed as limiting the scope of the invention, which is that as described in the appended claims.

### Example 1: Assessment of Solid Substrates as Germination Substrates

Mature spruce somatic embryos were prepared according to standard procedures.

The present inventors developed selection criteria for testing and evaluating a large number of different solid substrates for utility in the methods for somatic embryogenesis of spruce plants. These criteria included assessment of properties relates to germination, root anchoring, washability, tolerability to sterilization, etc.

In a first selection step, several substrates were tested with respect to germination of plant embryos. The substrates were ranked from 0-5 corresponding to the proportion of plant embryos that germinated successfully, wherein 0=0%, 1=1-20%; 2=21-40%; 3=41-60%; 4=61-80%; 5=>80%.

In a second selection step, substrates ranked as 1-5 in the first selection step were evaluated for root anchoring, i.e. an assessment of whether the plant embryo develops a root that anchors in the substrate. Analogous to the first selection step, the substrates were ranked from 0-5 corresponding to the proportion of plant embryos that anchored successfully, wherein 0=0%, 1=1-20%; 2=21-40%; 3=41-60%; 4=61-80%; 5=>80%.

The substrates were also evaluated for washability, i.e. how easily germination medium can be washed out of the substrate using water, and tolerability to sterilization.

The present inventors have also realized that in order, to use a solid substrate as a germination substrate in an automated method, the substrate is preferably both compressible and resilient. Several substrates were thus evaluated also for these properties.

It was found that a compressible and resilient solid substrate comprising a plurality of hydrophilic and biodegradable polymer fibres, such as polylactic acid, is most suitable as a germination substrate in the methods and systems for germination of a somatic plant embryo according to the invention.

One substrate useful as a germination substrate in the method according to the invention is commercially available under the brand name, “WhitePlug Sow” (Holland BioProducts, Nijmegen, The Netherlands). These plugs comprise a polylactic acid (PLA) stable fibre in combination with flakes of a super absorbent polymer of poly-potassium acrylic co-polymer, called bioflakes. The PLA fibre substrate can be used as a germination substrate in the present invention, either without the polymer flakes or in combination with such polymer flakes.

TABLE 1
Number of germinated and anchored spruce somatic embryos.

| | | Germi- | | An- | |
|---|---|---|---|---|---|
| | Total no | nated | Germi- | chored | An- |
| | plant | (No | nated | (No | chored |
| Substrate | embryos | embryos) | (%) | embryos) | (%) |
| 1. Oasis, GF11 | 90 | 60 | 75 | 33 | 37 |
| * | | | | | |
| 3. HP1, PLA | 60 | 55 | 92 | 45 | 75 |
| from 12 mm | | | | | |
| mats with Bio | | | | | |
| flakes | | | | | |
| 6. Rockwool 1, | 480 | 103 | 21 | 2 | 0.4 |
| 35 × 35 cubes | | | | | |
| 7. Rockwool 2, | 45 | 41 | 91 | 2 | 4 |
| Round | | | | | |
| cylinders | | | | | |
| 8. GrowFoam 1, | 53 | 46 | 87 | 0 | 0 |
| Type ++ | | | | | |

Suppliers of the tested growth substrates are GF11, Oasis oasis® GROWER SOLUTIONS, all HP material originate from Holland BioProducts, Nijmegen, The Netherlands. The Rockwool samples came from Grodan®, ROXUL Inc., Milton, Canada and GrowFoam 1 from Foamplant BV, Groningen, The Netherlands.

HP without bioflakes was also tested and gave very good germination. They do not differ much from the HP with bioflakes in terms of root anchoring.

### Example 2: Mechanical Properties of a Selected Group of Growth Substrates

Lasting deformation, maximum force and the return force were evaluated for 9 different growth substrates during and after a penetration of an 04 mm probe.

The following substrates were intended to be tested.
1. Oasis, GF11
3. HP1, PLA from 12 mm mats with Bio flakes
4. HP2, 3 off 4 mm thick mats of PLA without bio flakes
5. HP3, PLA with thick fibers without bio flakes
6. Rockwool 1, 35×35 cubes
7. Rockwool 2, Round cylinders
8. GrowFoam 1, Type++

The test samples were conditioned at (23±2°) C. and (50±5)% relative humidity for at least 24 h before the test. A Zwick Z1.0 universal testing machine was used, with a 50 N load cell. Temperature was 23.2° C. at 50.2% relative humidity.

The test was performed with a test probe of diameter 4 mm that was set to penetrate or compress the material 10 mm at a speed of 900 mm/minute. The test started at a preload of 0.01 N except for material 8 were a preload of 0.04 was set. After penetration or compression of the material a hold time of 5 seconds were set, after that the probe travelled up again at a speed of 3 mm/minute, during this time the force of the material were measured and when the force was close to 0.002 N the material had stopped its return. (lasting compression). After the test, the diameter of the holes was measured with a calliper.

The results are provided in Tables 2 and 3.

TABLE 2
Average results for maximum force and lasting
compression for the seven materials.

| | | Lasting | Hole size |
|---|---|---|---|
| | F max | compression | after test |
| Substrate | (N) | (mm) | (mm) |
| 1. GF 11 Oasis | 1.71 | 9.82 | 2.8 |
| 3. HP1 | 1.48 | 6.1 | N/A³ |
| 4. HP2 | 0.82 | 6.26 | N/A³ |
| 5. HP3 | 1.38 | 5.5 | N/A³ |
| 6. Rockwool 1 | 2.6 | N/A¹ | 2.6 |
| 7. Rockwool 2 | 2.88 | N/A¹ | 2.9 |
| 8. GrowFoam 1 | 4.43 | N/A¹ | N/A² |

¹The friction force of the probe was higher than the elevating force of the material.
²The material was only compressed but not penetrated.
³The material closed again after test.

TABLE 3
Force at depth 2, 3, 4 & 5 mm of penetration

| Substrate | 2 mm (N) | 3 mm (N) | 4 mm (N) | 5 mm (N) |
|---|---|---|---|---|
| 1. GF 11 Oasis | 0.93 | 1.29 | 1.4 | 1.47 |
| 3. HP1 | 0.16 | 0.26 | 0.39 | 0.54 |
| 4. HP2 | 0.13 | 0.20 | 0.28 | 0.38 |
| 5. HP3 | 0.07 | 0.13 | 0.22 | 0.34 |
| 6. Rockwool 1 | 0.97 | 1.43 | 1.69 | 1.94 |
| 7. Rockwool 2 | 1.60 | 2.12 | 2.37 | 2.50 |
| 8. GrowFoam 1 | 1.45 | 1.95 | 2.31 | 2.61 |

### Example 3: Water Retention Capacity

The materials evaluated in Example 2 were also evaluated in respect of Water Retention Capacity (WRC). To measure the Water Retention Capacity, a piece of the material of known dry weight (W_{d}) and volume (V) is soaked in a surplus of water and left in room temperature for one hour on a mesh to allow water to drain and then weighed with retained water to obtain a wet weight (W_{w}). The Water Retention Capacity is calculated as (W_{w}−W_{d})/V.

TABLE 3
Water Retention Capacity

| Substrate | WRC (g/cm³) | SD |
|---|---|---|
| 1. Oasis | 0.69 | 0.18 |
| 3. HP1 | 0.85 | 0.09 |
| 4. HP2 | 0.85 | 0.23 |
| 5. HP3 | 0.93 | 0.11 |
| 6. Rockwool 1 | 0.89 | 0.11 |
| 7. Rockwool 2 | 0.99 | 0.05 |
| 8. GrowFoam 1 | 0.32 | 0.16 |

### Example 4: Fibre Thickness of PLA

Microscopy check of the thickness of the HP1, 2 and 3 and Rockwool fibres was checked at two different magnifications. The HP fibres made of PLA is approximately 40 μm. Rockwool is about 5 to 10 μm.

### Example 5: Desiccation on Substrate or in Petri Dish

Mature spruce somatic embryos from two cell lines (A, B) of Pices abies (Norway spruce) were prepared according to standard procedure. To evaluate the feasibility of desiccation on growth substrate (PLA), a desiccation step was performed prior to germination on either a PLA fibre substrate or on in Petri dishes. For both substrates, desiccation was done under high humidity without direct contact between water and embryo. For Petri dishes, embryos were placed in empty top part of 6 cm Petri dish, in turn placed inside a 9 cm Petri dish. Around 3 ml of water was kept in the bigger Petri dish to maintain high humidity during desiccation. For PLA substrate, the embryo was placed on top of the PLA substrate and sterile water was flowed through the lower part of the substrate.

Germination of embryos desiccated on PLA was performed on either PLA substrate or in a Petri dish, whereas embryos desiccated in Petri dishes were only germinated in a Petri dish.

About 120 embryos per cell line were desiccated on PLA substrate. The desiccation was carried out for 2-3 weeks, under dark condition, with temperature of 20±2° C. After desiccation, half of the embryos were moved to a Petri dish containing appropriate germination medium for germination and the other half was germinated on same PLA substrate used for desiccation, after addition of appropriate germination medium.

As control, about 20 embryos per cell line were desiccated in Petri dish, placed inside a 9 cm Petri dish as above. After 3 weeks of control desiccation, the embryos were moved to Petri dish containing germination media for 6 weeks of germination.

The results are provided in Table 4.

TABLE 4

| | | Desiccation | Germination | | | | | % | % |
|---|---|---|---|---|---|---|---|---|---|
| Treatment | Cellline | on | on | Total | Root | Shoot | Both | Root | Shoot |
| T1 | A | PLA | PLA | 60 | 11 | 0 | 0 | 18.3 | 0.0 |
| T2 | A | PLA | Petridish | 57 | 28 | 7 | 4 | 49.1 | 12.3 |
| T3 | B | PLA | PLA | 58 | 16 | 21 | 7 | 27.6 | 36.2 |
| T4 | B | PLA | Petridish | 59 | 40 | 35 | 26 | 67.8 | 59.3 |
| T5 | A | Petridish | Petridish | 20 | 6 | 6 | 2 | 30.0 | 30.0 |
| T6 | B | Petridish | Petridish | 22 | 5 | 18 | 5 | 22.7 | 81.8 |

Desiccation in PLA give similar or improved percentage of roots as compared to the standard procedure of desiccation in Petri dish, indicating suitability of PLA substrate as both agent for desiccation and germination. This is surprising as combined desiccation and germination on other solid substrates commonly used for cultivation of plantlets has not been successful. The possibility to perform desiccation, germination and cultivation into a plantlet on a single substrate, without the need for mechanical transfer between substrates, significantly simplifies the automated process of obtaining plantlets from somatic plant embryos.

### REFERENCES

Belmonte, M. F. et al. (2005). Alterations of the glutathione redox state improve apical meristem structure and somatic embryo quality in white spruce (Picea glauca). Journal of experimental biology, 56(419), 2355-2364.
von Arnold et al. (2008). Spruce embryogenesis. In Suarez M. F., Bozhkov P. V. (Ed.), Plant Embryogenesis. Methods In Molecular Biology (pp. 31-47). Humana Press. doi:https://doi.org/10.1007/978-1-59745-273-1_3

1. A method for obtaining a plantlet from a plurality of somatic plant embryos by germination of a somatic plant embryo, comprising
bringing an opening of a nozzle of an embryo transfer device connected to a sub-pressure generator into contact with a plant embryo within the plurality of somatic plant embryos,
applying a sub-pressure to said opening to thereby attach the plant embryo to the nozzle by way of suction;
classifying the plant embryo as viable or non-viable;
providing a germination substrate;
optionally preparing a well in the germination substrate;
transferring the plant embryo to the germination substrate if classified as viable, optionally to said well; and
incubating said germination substrate under conditions suitable for somatic embryo germination, thereby obtaining a plantlet.

2. The method according to claim 1, wherein the classification step comprises capturing a digital image of at least one embryo and using a Machine Learning or an image analysis algorithm to classify the embryo as viable for use in subsequent steps of the method.

3. The method according to claim 1, wherein the transfer of the plant embryo to the germination substrate comprising bringing the plant embryo into contact with the germination substrate before detaching the plant embryo from the embryo transfer device.

4. The method according to claim 3, comprising bringing the plant embryo into contact with the germination substrate at an angle of 0°-30°, preferably 0°-10°, to a vertical axis.

5. The method according to claim 1, further comprising the steps
adding germination medium to the germination substrate; and
washing out the germination medium after 1 week or more, such as after 2 weeks or more, such as after 15 weeks or less, such as after 10 weeks or less, such as after 6 weeks or less, and/or further comprising transferring the germination substrate with the plantlet to a soil.

6. (canceled)

7. The method according to claim 1, comprising transferring the plant embryo to a well in the germination substrate with the root forming end down and in contact with substrate, wherein the well optionally has a diameter of 2-6 mm and a depth of 3 mm or more.

8. The method according to claim 7, wherein the diameter of the well is reduced after transfer of the plant embryo to the well, such as by allowing the surrounding germination substrate to expand or by applying pressure to at least part of the surrounding germination substrate.

9. The method according to claim 1, further comprising at least one of the following additional steps
desiccating the embryo on the germination substrate prior to incubations under conditions suitable for somatic embryo germination;
adding a granular top-dressing material on top of the germination substrate to at least partially cover the plant embryo;
removing excess fluid from the selected plant embryo by suction through the nozzle of the embryo transfer device; and
growing and acclimating the plantlet ex vitro in the solid germination substrate.

10. The method according to claim 1, comprising use of a compressible and resilient solid substrate comprising a plurality of hydrophilic and biodegradable polymer fibres as a germination substrate.

11. The method according to claim 10, wherein the solid substrate can be compressed at least 3 mm by applying a pressure of less than 0.08 N/mm², such as at least 3 mm by applying a pressure of less than 0.04 N/mm², and/or
wherein the solid substrate is free of loose adhesive material, and/or wherein the solid substrate is substantially free of particulate matter of less than 0.015 mm width and 2 mm length, and/or
wherein the solid substrate has a water retention capacity of 0.3-1.2 cm3, such as 0.5-1.2 g/cm3, preferably 0.7-1.0 g/cm3, and/or
wherein the polymer fibres are non-porous, and/or
wherein the polymer fibres are constituted of polylactic acid, and/or
wherein the compressible and resilient solid substrate is used as a desiccation substrate and a germination substrate.

12. (canceled)

13. (canceled)

14. (canceled)

15. (canceled)

16. (canceled)

17. A system for obtaining a plantlet from a plurality of somatic plant embryos, comprising
an embryo holding container configured to hold a plurality of plant embryos;
an embryo selection unit configured to classify a plant embryo of the plurality of plant embryos as viable or non-viable;
an embryo transfer device connected to sub-pressure generator with a nozzle having a tip surface comprising an opening in fluid contact with the sub-pressure generator; and
at least one germination substrate
wherein the embryo transfer device is arranged on a robotic arm configured to bring the opening of the nozzle in contact with a plant embryo to facilitate initial attachment of the plant embryo to the nozzle through application of a sub-pressure to the opening, and transfer a plant embryo classified as viable from said embryo holding container to said germination substrate.

18. The system according to claim 17, wherein the selection unit comprises a device for capturing a digital image of the plurality of plant embryos and a computer having installed thereon a Machine Learning unit trained to select a viable plant embryo or an image analysis algorithm configured to select a viable plant embryo, and/or
wherein the embryo transfer device is configured to bring the plant embryo into contact with the germination substrate at an angle of 0°-30°, preferably 0°-10°, to a vertical axis, and/or.
wherein the nozzle tip has an outer diameter of less than 5 mm, and/or.
wherein the nozzle opening is provided within a groove in the nozzle tip surface, wherein said groove is adapted to accommodate a somatic plant embryo of a plant species with which the system is configured to be used.

19. (canceled)

20. (canceled)

21. (canceled)

22. The system according to claim 17, wherein the nozzle comprises an outer tube and an inner tube protruding from the outer tube and ending with the nozzle tip surface at a distal end of the nozzle and the proximal end of the inner tube being connected to the sub-pressure generator, and the nozzle being configured so that positive pressure can be applied to the space between the outer tube and the inner tube to provide an air stream towards the distal end of the nozzle.

23. The system according to claim 22, wherein the wall of the outer tube is provided with one or more through-holes for application of positive pressure.

24. The system according to claim 17, wherein the germination substrate is a compressible and resilient solid substrate comprising a plurality of hydrophilic and biodegradable polymer fibres as a germination substrate.

25. The system according to claim 24, wherein the solid substrate can be compressed at least 3 mm by applying a pressure of less than 0.08 N/mm², such as at least 3 mm by applying a pressure of less than 0.04 N/mm², and/or.
wherein the solid substrate is free of loose adhesive material, and/or wherein the solid substrate is substantially free of particulate matter of less than 0.015 mm width and 2 mm length, and/or.
wherein the solid substrate has a water retention capacity of 0.3-1.2 cm³, such as 0.5-1.2 g/cm³, preferably 0.7-1.0 g/cm³, and/or.
wherein the polymer fibres are non-porous, and/or.
wherein the polymer fibres are constituted of polylactic acid, and/or.
wherein the compressible and resilient solid substrate is used as a desiccation substrate and a germination substrate.

26. (canceled)

27. (canceled)

28. (canceled)

29. (canceled)

30. (canceled)

31. A method of germinating a somatic plant embryo comprising using a compressible and resilient solid substrate comprising a plurality of hydrophilic and biodegradable polymer fibres as a germination substrate.

32. The method according to claim 31, wherein the solid substrate can be compressed at least 3 mm by applying a pressure of less than 0.08 N/mm², such as at least 3 mm by applying a pressure of less than 0.04 N/mm² and/or.
wherein the solid substrate is free of loose adhesive material, and/or wherein the solid substrate is substantially free of particulate matter of less than 0.015 mm width and 2 mm length, and/or.
wherein the solid substrate has a water retention capacity of 0.3-1.2 cm3, such as 0.5-1.2 g/cm3, preferably 0.7-1.0 g/cm3, and/or.
wherein the polymer fibres are non-porous, and/or.
wherein the polymer fibres are constituted of polylactic acid, and/or.
wherein the compressible and resilient solid substrate is used as a desiccation substrate and a germination substrate.

33. (canceled)

34. (canceled)

35. (canceled)

36. (canceled)

37. (canceled)

38. A method for obtaining a plantlet from a somatic plant embryo, comprising
placing a mature somatic plant embryo on a compressible and resilient solid substrate comprising a plurality of hydrophilic and biodegradable polymer fibres;
desiccating said somatic plant embryo;
adding a germination medium to the solid substrate; and
incubating the desiccated somatic plant embryo on the solid substrate under conditions suitable for somatic embryo germination, thereby obtaining a plantlet.

39. The method according to claim 38 wherein the compressible and resilient solid substrate comprising a plurality of hydrophilic and biodegradable polymer fibres is used as a desiccation substrate and a germination substrate.
